(19) United States
(12) Patent Application Publication
(10) Pub. No.: US 2022/0371394 A1
(43) Pub. Date: Nov. 24, 2022

(54) METHOD AND SYSTEM FOR REDUCING A PROBABILITY OF TRANSMITTING PATHOGENS IN A VEHICLE

(71) Applicant: Ford Global Technologies, LLC, Dearborn, MI (US)

(72) Inventors: Rainer Vogt, Aachen (DE); Volker Scheer, Roetgen (DE)

(73) Assignee: Ford Global Technologies, LLC, Dearborn, MI (US)

(21) Appl. No.: 17/658,844

(22) Filed: Apr. 12, 2022

(30) Foreign Application Priority Data
May 18, 2021 (DE) 102021112779.3

(51) Int. Cl. B60H 1/00

(52) U.S. Cl. CPC B60H 1/008; B60H 1/00757; B60H 1/00742

(57) ABSTRACT

A method and a system for reducing a probability of transmitting pathogens between vehicle occupants located in an interior of a vehicle is disclosed. A hazardous situation present in a vehicle interior of the vehicle is recognized, which is associated with an elevated probability of transmitting pathogens, namely by registering a vehicle occupant-related event associated with the elevated probability of transmitting pathogens, and/or by registering a current pathogen load in the vehicle interior, and/or on the basis of an instruction provided by a vehicle occupant or externally, from which the elevated probability of transmitting pathogens and thus the hazardous situation can be concluded. In the event of a recognized hazardous situation, a measured for reducing the probability of transmitting pathogens in the vehicle interior is initiated.

### CROSS-REFERENCE TO RELATED APPLICATIONS

The present application claims priority to and the benefit of German Application No. DE 102021112779.3, filed May 18, 2021, which is hereby incorporated by reference herein in its entirety.

### FIELD

The present invention relates to a method for reducing a probability of transmitting pathogens between vehicle occupants located in a vehicle interior of a vehicle. Furthermore, the present invention relates to a system for executing a method according to the invention, wherein the system is implemented in a vehicle. Finally, the present invention comprises a vehicle, in particular a motor vehicle, comprising a system according to the invention.

### BACKGROUND

Pathogens such as germs, microorganisms, viruses, viroids, bacteria, spores, cells, algae, bacteria, parasites, fungi, prions, protists, subcellular pathogens, or the like can be transmitted between human or animal living beings in different ways. One possible transmission path relates to smear infections. Furthermore, pathogens can be transmitted via the air, for example via droplet transmissions, aerosol transmissions, or other types of transmissions of infectious material. The infectious material can be composed for example of the pathogens and further substances (for example liquids or secretions from the oral-pharyngeal-nasal cavity of a living being). The infectious material can be transmitted in the form of particles, particle agglomerates, or droplets of different sizes.

In the course of the corona pandemic arising worldwide in the year 2020, it became known that certain pathogens (in this case viruses) can be transmitted particularly easily via the air between living beings, e.g., from human to human, from human to animal, from animal to human, and from animal to animal. In particular droplet and aerosol transmissions play an essential role here. In enclosed spaces, and at short distances between the living beings, the risk of such a transmission is elevated. It is also the case that in vehicles equipped with a vehicle cabin for vehicle occupants (a closed space in which the vehicle occupants are generally positioned with little distance in relation to one another), the risk of transmission of the pathogens via the cabin air can be increased. Measures for reducing the probability of transmitting pathogens in vehicles are accordingly of particular interest and can represent a reasonable means for reducing risks of infection.

Both large droplets having a diameter of >10 μm (so-called respiratory droplets) and also aerosol particles (i.e. <5-10 μm diameter) can be triggers for the transmission of respiratory illnesses (WHO, 2020). Although the infection pathways of the novel coronavirus (associated respiratory illness: COVID-19) have been scientifically discussed, the WHO states that the novel coronavirus is transmitted according to present knowledge between humans inter alia in a respiratory manner via droplet transmissions. According to the WHO, droplet transmission can take place when a person is in close contact (within 1 m) with another person, one of the persons displays respiratory systems (for example coughing or sneezing), and there is therefore a risk of coming into contact with potentially infectious respiratory droplets via mucosa (mouth and nose) or conjunctiva (eyes). Transmission via aerosol particles is also considered to be significant.

In the vehicle interior (for example a vehicle cabin), the vehicle occupants are naturally close to one another, therefore the risk of infection at such a location is particularly elevated in the event of coughing or sneezing. It is known that the exposure time with respect to infectious material is an important factor. Exchanging the air located in the vehicle interior with fresh air, selecting matching airflow directions, and filtering out pathogens could represent suitable measures for reducing the probability of pathogen transmission if the source of the pathogens, the propagation direction thereof in the vehicle interior, and the position of the vehicle occupants to be protected were known at the time of the measure.

### SUMMARY

Accordingly, the present invention is based on the object of providing a method, system, and vehicle, using which the risk of infection for vehicle occupants located in a vehicle by pathogens can be reduced.

To achieve this object, a method having the features of claim 1, a system having the features of claim 16, and a vehicle having the features of claim 17 are proposed.

The invention firstly relates to a method for reducing a probability of transmitting pathogens between vehicle occupants located in a vehicle interior of a vehicle, in which
a. a hazardous situation, which is associated with an elevated probability of transmitting pathogens, present in a vehicle interior of the vehicle is recognized, namely
   i. by registering a vehicle occupant-related event associated with the elevated probability of transmitting pathogens, and/or
   ii. by registering a current pathogen load in the vehicle interior and subsequently checking whether the registered current pathogen load is above a predefined threshold pathogen load, wherein the hazardous situation is present in the case of a current pathogen load lying above the threshold pathogen load, and/or
   iii. on the basis of an instruction provided by vehicle occupant or externally, which permits the elevated probability of transmitting pathogens and thus the hazardous situation to be concluded;
b. an event-related, vehicle occupant-related, and/or vehicle-related parameter or a parameter tuple is ascertained;
c. in the event of a recognized hazardous situation, a measure is initiated to reduce the probability of transmitting pathogens in the vehicle interior taking account of the ascertained parameter or parameter tuple.

A “vehicle” can be understood in the meaning of the invention as a wheeled motor vehicle such as a passenger vehicle, a truck, a bus, an omnibus, a transport vehicle, a van, a construction site vehicle, an agricultural vehicle, a utility vehicle, or the like. The vehicle can also be a rail vehicle such as a local, regional, or long-distance train, a high-speed train, a streetcar, a tram, a subway, or the like. The “vehicle” can involve public personal transport vehicles or private vehicles. The invention can fundamentally be used in all vehicles which have a vehicle interior (for example a passenger cabin), in which multiple living beings (humans, animals) can be conveyed, in which living beings are thus positioned at relatively short distances (for example <1.5 m or 1 m) relative to one another. “Vehicle occupants” can be understood as drivers, front passengers, passengers, animals, etc.

The mentioned hazardous situation associated with an elevated probability of transmitting pathogens can be a situation in which multiple (at least two) vehicle occupants are in the vehicle interior. Here, the vehicle can be in a travel state, a holding state (for example with running engine), a parked state, or at a standstill (with or without running engine).

The probability of transmitting pathogens predominantly relates to transmission of aerosols or droplets via the air located in the vehicle interior. The presence of an “elevated” probability of transmitting pathogens can be based on different criteria or events. Depending on the criterion or event, different threshold values can be defined, which, when they are exceeded or not reached, present an elevated probability of transmitting pathogens. “Pathogens” can be understood—as mentioned at the outset—as e.g. germs, microorganisms, viruses, viroids, bacteria, spores, cells, algae, bacteria, parasites, fungi, prions, protists, subcellular pathogens, or the like.

It is to be emphasized that any data evaluations performed in the scope of the invention can be performed in a software-based manner, a computer-assisted manner, algorithmically, or on the basis of artificial intelligence.

According to the invention, the elevated probability of transmitting pathogens can be recognized by registering a vehicle occupant-related event associated with the elevated probability of transmitting pathogens. The vehicle occupant-related event can be a behavior (for example a movement) established in the behavior of one or more vehicle occupants (whether human or animal) or a corresponding physical event (coughing, sneezing, loud speaking, change of the body temperature, etc.). The event can be registered in particular by one or more sensors arranged in or on the vehicle. Here, the event can occur spontaneously and have a short chronological duration, it can be as it were a repeatedly or continuously prolonged event. “Registering” can be understood for example as detecting, measuring, or recording using a registration means. A registration means can accordingly be a detector, a sensor, a measuring device, a recording device, or a plurality of the above-mentioned items, for example. The step of “registering” can as it were already include a data evaluation of detected, measured, or recorded measurement data. A plurality of the mentioned vehicle occupant-related events can also be registered and an elevated probability of transmitting pathogens based thereon can then be recognized. The step of “recognition” can also include a data evaluation of registered data.

Additionally or alternatively, the elevated probability of transmitting pathogens can be recognized by registering a current pathogen load in the vehicle interior and subsequently checking whether the registered current pathogen load is above a predefined threshold pathogen load, wherein the hazardous situation is present in the event of a current pathogen load lying above the threshold pathogen load. The current pathogen load can be registered using a pathogen load registration means. The pathogen load can be ascertained continuously, at defined (settable) time intervals, or spontaneously. The procedure of “registering a current pathogen load” can in particular be a measurement of the pathogen load. This can be a direct measurement of pathogen-specific features (e.g., chemical, biological, physical features) from which a load (for example in the meaning of a concentration, thus e.g. a number, density, or mass of pathogens per unit of volume of room air) may be concluded. Proportionality measurements, i.e. measurements from which a determined load (for example in the meaning of a concentration, thus e.g. a number, density, or mass of pathogens per unit of volume of room air) can be concluded indirectly can also be suitable for “registering” a pathogen load. The threshold pathogen load can be a limit pathogen load, which, when it is exceeded, means an elevated probability of transmitting pathogens can be present. The check can be carried out using a checking means. The threshold pathogen load can be predefined and/or updated at defined time intervals (or spontaneously), for example, by a vehicle producer, a vehicle operator, or a vehicle owner. The threshold pathogen load can also be predefined or updated during regular repair shop visits (for example during the inspection of a vehicle). The threshold pathogen load can be stored on a vehicle-side computing and monitoring unit. The computing and monitoring unit can be assigned a corresponding data memory. The same can take place on an external computing and monitoring unit connected in terms of communications technology to the vehicle-side computing and monitoring unit.

Additionally or alternatively, the elevated probability of transmitting pathogens can be recognized on the basis of an instruction provided by a vehicle occupant or externally, from which the elevated probability of transmitting pathogens and thus the hazardous situation may be concluded. The instruction can be received by a corresponding receiving means. An instruction provided by a vehicle occupant can be for example a manual input of a vehicle occupant in a vehicle-side input system. The input system can be a keyboard, a touchscreen, a joystick, a push button, or another input means which is connected to the vehicle-side or external computing and monitoring unit (in a wireless or wired manner). A smart phone, tablet computer, or the like, which is connected in terms of communications technology to the vehicle-side or external computing and monitoring unit, can also be understood as an input means. An item of information, according to which a vehicle occupant has received a positive test result with respect to an infection with a pathogen, can underlie the instruction. A hazardous situation relating to an elevated probability of transmitting pathogens can be connected to such a test result. The fact of the positive test result can also be transmitted directly to the vehicle-side computing and monitoring unit or to an external computing and monitoring unit connected to the vehicle-side computing and monitoring unit. Other information can also underlie such an instruction, for example a generally worsened general state (for example discomfort) of a vehicle occupant.

According to the method, an event-related, vehicle occupant-related, and/or vehicle-related parameter or a parameter tuple is ascertained. This means that data registered (i.e., detected, measured, or recorded) during the registration of the event can be used to ascertain (e.g., calculate, derive, estimate) an event-related parameter or a plurality of such parameters (parameter tuple). Individual parameters or parameter tuples can also be ascertained from vehicle occupant-related data or items of information (whether they are measured, predefined, or estimated items of information). This also applies to vehicle-related parameters which can predominantly be operating parameters of certain vehicle-side functional units. A parameter tuple (thus a plurality of parameters) can exclusively comprise event-related, vehicle occupant-related, or vehicle-related parameters, or a combination of the above-mentioned parameters. The parameter tuple can also comprise further parameters (not mentioned here). Furthermore, the ascertainment of a plurality of parameter tuples can be provided. Parameter tuples can be generated in particular in the form of data vectors or matrices.

According to the method, in the event of a recognized hazardous situation, a measure is initiated to reduce the probability of transmitting pathogens in the vehicle interior taking consideration of the ascertained parameter or parameter tuple. The initiation of the measure preferably takes place automatically, for example via a vehicle-side computing and monitoring unit or an external computing and monitoring unit connected to the vehicle-side computing and monitoring unit. The probability of transmitting pathogens between the vehicle occupants is reduced and occupant protection from an infection with the pathogen is elevated by the measure. This procedure opens up an increase of the safety while simultaneously allowing continued operation of the vehicle.

Using the method according to the invention, it can be recognized, inter alia, when a person coughs, sneezes, speaks with high intensity, or is infectious. In the case of such a recognition, the position of the vehicle occupants, the location of the source of infectious material, and the propagation direction of the infectious material can be ascertained with the aid of sensors and items of vehicle system information present in the vehicle. In a second step, measures can be taken to reduce a probability of transmitting pathogens, for example changing the airflow settings, opening the windows, or best air purification measures.

It is to be noted that the object according to the invention is also achieved by a system for carrying out the method according to the invention, wherein the system is implemented in a vehicle, comprising
a. establishing means for establishing a hazardous situation present in a vehicle interior of the vehicle, which is associated with an elevated probability of transmitting pathogens, namely
   i. registration means for registering a vehicle occupant-related event associated with the elevated probability of transmitting pathogens, and/or
   ii. pathogen load registration means for registering a current pathogen load in the vehicle interior and checking means for checking whether the registered current pathogen load is above a predefined threshold pathogen load, wherein the hazardous situation is present in the event of a current pathogen load lying above the threshold pathogen load, and/or
   iii. receiving means for receiving an instruction provided by a vehicle occupant or externally, from which the elevated probability of transmitting pathogens and thus the hazardous situation can be concluded;
b. means for ascertaining an event-related, vehicle occupant-related, and/or vehicle-related parameter or a parameter tuple;
c. means for initiating a measure for reducing the probability of transmitting pathogens in the vehicle interior taking consideration of the ascertained parameter or parameter tuple in the event of an established hazardous situation.

The mentioned establishing means are preferably implemented in a vehicle-side computing and monitoring unit. Furthermore, the establishing means can be implemented in an external computing and monitoring unit, wherein the external computing and monitoring unit is (in particular wirelessly) connected in terms of communications and signaling technology to the vehicle-side computing and monitoring unit. The establishing means can be software-based, algorithmic, or AI (artificial intelligence)-based establishing means.

Furthermore, it is to be noted that the object underlying the invention is achieved by a vehicle, in particular a motor vehicle. The vehicle comprises a system according to the invention.

It is to be noted that the features set forth individually in the claims can be combined with one another in any technically reasonable manner and disclose further embodiments of the invention. The description additionally characterizes and specifies the invention in particular in conjunction with the figures. The features described in conjunction with the method according to the invention can also be advantageous embodiments of the system or vehicle according to the invention and vice versa.

It is furthermore to be noted that a conjunction “and/or” used herein, which is between two features and links them to one another, is always to be interpreted to mean that in a first embodiment of the subject matter according to the invention only the first feature can be present, in a second embodiment only the second feature can be present, and in a third embodiment both the first and the second feature can be present.

Further advantageous embodiments of a method according to the invention result from the features specified in the dependent claims and described hereinafter. The features specified in the dependent claims are also described hereinafter. It is to be emphasized here that the features described hereinafter can readily also be advantageous embodiment features of a system or vehicle according to the invention. To avoid repetitions, the features under discussion are only described hereinafter with reference to the method according to the invention.

According to a first embodiment of the invention, it can be provided in a method according to the invention that the method is carried out using a vehicle-side computing and monitoring unit, in particular an onboard computer, or an external computing and monitoring unit connected in terms of communications technology to the vehicle-side computing and monitoring unit. The above-mentioned means, in particular the establishing means, registration means, pathogen load registration means, checking means, receiving means, means for ascertaining the event-related, vehicle occupant-related, and/or vehicle-related parameter or the parameter tuple, and the means for initiating a measure for reducing the probability of transmitting pathogens can be embodied on the vehicle-side computing and monitoring unit and/or can be connected thereto in terms of signaling technology. The same can also apply to the external computing and monitoring unit, to which the mentioned means can be connected in terms of signaling technology and/or on which they can be embodied. The means can in some cases be sensor devices, measuring devices, or recording devices, but in some cases also software, programs, or algorithms.

If they are sensor devices, measuring devices, or recording devices, these can each additionally have microcontrollers or processor units for preprocessing.

According to a further embodiment of the invention, it can be provided in a method according to the invention that an event linked to an elevated substance emission via the oral-pharyngeal-nasal cavity of a vehicle occupant is registered as the vehicle occupant-related event. Greatly varying substances can be discharged directly to the surroundings via the oral-pharyngeal-nasal cavity of a living being, i.e., without the substances having to traverse a barrier. Accordingly, such a substance emission is generally associated with a probability of transmitting pathogens. This is because emitted substances (e.g., aerosols, particles, droplets) can be distributed easily in the air of the vehicle interior and inhaled by other vehicle occupants. In the case of direct emission of substances in the direction of other vehicle occupants, they can even come directly into contact with the mucosa of the oral-nasal-pharyngeal cavity or the ocular conjunctiva. Direct inhalation of such substances can also thus be intensified.

According to a further embodiment of the invention, it can be provided in a method according to the invention that the event is a coughing event, a sneezing event, a speech event, a voice event, a singing event, a respiratory event, a movement event, or a vomiting event, in which the substance is emitted. A coughing event can be understood as a cough reflex, a dry cough, coughing up phlegm, or the like. A respiratory event can be understood, for example, as a hiccup event or another physical mechanism (in which substances are emitted via the oral-nasal-pharyngeal cavity). A speech event can be represented as a vehicle occupant-related event having elevated substance emission if a certain speech intensity (volume) is exceeded. When speaking at high volume, the oral movements are intensified and thus the risk of an elevated substance emission is automatically elevated. An elevated substance emission can also be present in certain pitches, which can be understood in the present terminology as a voice event. A singing event relates to the singing (or rapping) of a song or text. A movement event can relate to a certain movement pattern, a movement intensity, or the like, of vehicle occupants. Two fighting (or playing) children in a vehicle, who grab each other and/or come into close contact, is mentioned as an example. The probability for a substance emission of infectious material via the oral-pharyngeal-nasal cavity can be increased by the increased movement activity. A vomiting event can mean the emesis (throwing up) of a vehicle occupant. Infectious material can also be discharged from a vehicle occupant to the surroundings during such an event.

It can be provided that the coughing event, the sneezing event, the speech event, the voice event, the singing event, or the respiratory event is to be classified as such an event only above a certain intensity (for example volume), duration, or frequency. This can mean, for example, that light throat clearing is not classified as a coughing event, but forcefully coughing up phlegm is. The same can apply to speaking at low volume, singing at low volume and over a short duration, etc. This can also apply to other respiratory events.

According to a further embodiment of the invention, it can be provided in a method according to the invention that an elevated body temperature of a vehicle occupant is registered as the vehicle occupant-related event. An elevated body temperature can indicate an infection with a pathogen. For example, fever is associated with an often significantly elevated body temperature. Many types of infectious diseases are accompanied by fever, because of which an elevated body temperature or fever can represent an indication of an elevated probability of transmitting pathogens. An elevated body temperature or fever indicates a strengthened defensive response of the body, for example to defend from pathogens. The probability that a vehicle occupant (having elevated body temperature or fever) located in a vehicle will transmit pathogens to other vehicle occupants is thus often elevated. Therefore, an elevated body temperature or fever can be an indicator of an elevated probability of transmitting pathogens and thus a hazardous situation. The body temperature can be ascertained by means of temperature measuring units (e.g., contactless fever thermometers, contact fever thermometers, thermal imaging cameras, infrared sensors, or the like) provided in the vehicle.

According to a further embodiment of the invention, it can be provided in a method according to the invention that the event is registered using one or more sensor(s), in particular one or more acoustic sensor(s), one or more optical sensor(s), one or more movement sensor(s), one or more thermal sensor(s), one or more radiation sensor(s), one or more seat sensor(s), one or more weight sensors arranged on a vehicle seat, and/or one or more touch sensor(s). The mentioned sensors relate in particular to registering the vehicle occupant-related event. If multiple sensors (of the same or different types: thus for example multiple acoustic sensors or one acoustic and one optical sensor) are used, these can thus be arranged at different spatial positions in the vehicle, in particular the vehicle interior. This enables hazardous situations relating to an elevated probability of transmitting pathogens to be able to be ascertained reliably at different points of the vehicle interior.

With respect to the above-mentioned sensors, a vehicle-side computing and monitoring unit or an external computing and monitoring unit connected in terms of communications technology to the vehicle-side computing and monitoring unit can have characteristic parameters, characteristic data, boundary conditions, evaluation criteria, or the like predefined to it, which are compared to the registered (and possibly evaluated in a certain aspect) measurement data and are compared with respect to the presence of a vehicle occupant-related event associated with an elevated probability of transmitting pathogens. The predefined characteristic parameters, characteristic data, boundary conditions, evaluation criteria, or the like provide assessment scales for recognizing a vehicle occupant-related event. The qualitative fulfillment of one or more characteristic parameters, characteristic data, boundary conditions, evaluation criteria, or the like can indicate the presence of a vehicle occupant-related event associated with an elevated probability of transmitting pathogens. However, exceeding a limit value defined by the mentioned characteristic parameters, characteristic data, boundary conditions, evaluation criteria, or the like can also indicate the presence of the vehicle occupant-related event associated with an elevated probability of transmitting pathogens. The same also applies to the presence of certain (predefined) curves of time-resolved or location-resolved recorded measurement data.

An “acoustic sensor” can be understood in particular as a microphone or microphone system. This can have an amplification or pre-amplification unit or can be connected thereto. Using the acoustic sensor, noises such as sounds, voices, singing, and the like and associated noise parameters of vehicle occupants can be registered and recorded. The acoustic sensor is suitable for registering noise parameters such as amplitudes (intensities, level), frequencies, and the chronological duration of the noises. The registration of time-resolved or location-resolved noise signals is thus enabled, because of which certain chronological noise patterns (for example amplitude curves) can also be recorded using the acoustic sensor and can provide a noise parameter. Moreover, positional locating of noises and the determination of location-resolved noise parameters is enabled by a three-dimensional distribution of multiple acoustic sensors in the vehicle interior. On the basis of predefined noise parameters or noise data such as limit values for noise amplitudes, predefined patterns of chronological noise curves, or the like, deviations of the noise data or noise parameters actually registered using the acoustic sensor from the predefined noise parameters, patterns, or data can be established. In the event of a deviation going beyond a predetermined amount, an elevated probability of transmitting pathogens can thus be concluded. Critical limit values, limit value ranges, pattern curves, or the like can be predefined here, upon the exceeding or reaching of which it is possible to presume an elevated probability of transmitting pathogens. The predefined noise parameters or noise data can be ascertained experimentally or in the course of simulations.

An optical sensor can be understood, for example, as a sensor based on a reflective principle, but also a sensor system designed like a light barrier. In a sensor system designed like a light barrier, one or more light barriers can be arranged in the vehicle interior. In a reflective optical sensor, light or radiation can be emitted by a radiation source. Light or radiation reflected from a target object, thus for example from a body part of a vehicle occupant or a vehicle part, is then registered by the reflective optical sensor. Inter alia, the change of the light or radiation intensity (between emitted light or radiation and light or radiation received after reflection) can be ascertained here. The light or radiation intensity can vary, for example depending on the seat position (or positioning of the body or body parts) of the vehicle occupant, i.e., his distance to the sensor. Items of information about the seat position, movements, etc. of a vehicle occupant can be derived from the measurement data registered using the optical sensor, which items of information can indicate for example a certain vehicle occupant-related event, which is associated with an elevated probability of transmitting pathogens. Movements of vehicle occupants, which can indicate a certain vehicle occupant-related event, can also be registered by means of a light barrier-based sensor system. To determine a vehicle occupant-related event, data registered using the one or more optical sensors can be evaluated from a spatial and chronological perspective.

A movement sensor can be understood as a sensor using which a qualitative presence of a movement (i.e., the presence of a movement) can be recognized. A movement pattern, a movement sequence, or the like can also be registered using such a sensor. An acceleration sensor, for example a three-dimensional acceleration sensor, can also be regarded as a movement sensor. Movements can be registered from both a spatial and also chronological perspective using a movement sensor. A certain movement, a certain movement pattern, or a certain movement sequence can indicate the presence of a vehicle occupant-related event. Such movements can be predefined by the user, by the producer, or in another way and stored on the vehicle-side computing and monitoring unit or the external computing and monitoring unit connected in terms of communications technology to the vehicle-side computing and monitoring unit.

A thermal sensor can be understood for example as a thermal imaging camera or another type of temperature sensor. The thermal sensor can be a sensor which records the items of temperature information of the vehicle interior spatially, for example two-dimensionally or multidimensionally, or only registers items of temperature information at certain predefined spatial points. A thermal sensor can be based on infrared technology. Since the body temperature of the vehicle occupants generally differs from the vehicle interior, body silhouettes can be recorded by means of a thermal imaging camera. Certain movements, which permit an elevated probability of transmitting pathogens to be concluded, can also be registered by means of a thermal imaging camera. In addition, a thermal sensor or a thermal imaging camera can register an elevated body temperature of a vehicle occupant. A body temperature of a vehicle occupant elevated in comparison to the normal temperature may also permit an infection with a pathogen to be concluded. An increased risk of infection, thus an increased probability of transmitting pathogens, is then often linked thereto.

A radiation sensor can be understood as a sensor based on a radiation emission at a predefined wavelength or a wavelength range, in particular an ultrasonic sensor, infrared sensor, terahertz sensor, or microwave sensor. It can be provided here that radiation is emitted in a predefined frequency pattern, a predefined pulse sequence, or the like by the radiation sensor. Radiation sensors can be used for location-resolved and time-resolved measurement. A continuous (chronological) sequence of spatially (two-dimensionally or three-dimensionally) registered data can also be recorded using one or more radiation sensors. Using such sensors, vehicle occupant-related events, for example, coughing, sneezing, or the like can be registered, which as vehicle occupant-related events can be linked to an elevated probability of transmitting pathogens. Observations of the vehicle interior and thus of the vehicle occupants are possible using radiation sensors. Here, the vehicle occupants can be irradiated using radiation of a certain wavelength. The radiation reflected from the vehicle occupants can then be registered using a suitable sensor and evaluated with respect to establishing predetermined indicators (runtime, runtime delay, amplitude curve, phase curve, or the like) for the presence of a vehicle occupant-specific event linked to an elevated probability of transmitting pathogens.

A seat sensor can be understood as a sensor which reflects the occupancy of a seat in the vehicle interior by a vehicle occupant (i.e., it can be established by means of the seat sensor whether a vehicle occupant is seated on a vehicle seat). One or more seat sensors can be arranged on one seat. A seat position of a vehicle occupant can be ascertained by means of the seat sensor/seat sensors. The seat position of a vehicle occupant can supply important items of information on the propagation direction of pathogens upon the presence of a vehicle occupant-related event.

Seat sensors can accordingly be used in particular in combination with further sensors. Furthermore, one or more weight sensors and/or one or more touch sensor(s) can be arranged in or on the vehicle seats. These can supply items of information on a possible seat position and thus items of information with respect to a propagation direction of pathogens.

According to a further embodiment of the invention, it can be provided in a method according to the invention that the current pathogen load is registered by a sensor arranged in the vehicle for determining a pathogen load in a room air sample or a room volume element from the vehicle interior. A plurality of the mentioned sensors can also be provided in the vehicle, where the sensors can be arranged at different spatial points in the vehicle, in particular in the vehicle interior. Multiple sensors for registering different pathogen types (for example different viruses) can also be arranged in the vehicle. The sensors can be supplied with a room air sample (including droplets, aerosols, particles, or the like contained therein) via a corresponding device (for example a suction device), so that the room air sample can be examined in the sensors with respect to the presence of pathogens or a certain pathogen concentration. The ascertained pathogen load can be output via an output means and passed on to the vehicle-side computing and monitoring unit. The mentioned sensor for determining a pathogen load provides a pathogen load registration means.

According to a further embodiment of the invention, it can be provided in a method according to the invention that the instruction is based on a notification about a positive test of a pathogen infection of a vehicle occupant, wherein the instruction is transmitted in the course of a manual input of the vehicle occupant or automatically to the vehicle-side computing and monitoring unit. A positive test of a pathogen infection of a vehicle occupant (the test can be carried out for example by a specialized laboratory or as a rapid test) can inevitably be linked to an elevated probability of transmitting pathogens. This is because infected persons can tend per se to emit increased numbers of pathogens and—in the case of a vehicle—discharge them to the vehicle interior. It is accordingly advantageous that if a vehicle occupant is made aware of a possible infection with a pathogen (for example via a message on a mobile terminal, an app, or the like), a possibility is provided of effectuating an adjustment of certain vehicle settings or other measures to reduce the probability of transmitting pathogens in the vehicle interior. A manual input can be understood as the actuation of an input means, for example a switch in the vehicle (e.g., a button, switching lever, switching surface) or an input via a keyboard, a touchscreen, a speech input, or the like. The input means can be connected to a receiving means, wherein the receiving means can be a component of the vehicle-side computing and monitoring unit or the external computing and monitoring unit. Automatic transmission of the instruction, for example via a mobile terminal or an external server, to the vehicle-side computing and monitoring unit or the external computing and monitoring unit can also be provided. For example, as a result of a positive test for an infection of a vehicle occupant with a certain pathogen, here a corresponding notification can automatically be transmitted from a test laboratory to a mobile terminal of the vehicle occupant (for example an app operated on the terminal). The mobile terminal can in turn automatically be connected to the vehicle-side or external computing and monitoring unit and automatically pass on the corresponding data thereto.

According to a further embodiment of the invention, it can be provided in a method according to the invention that the event-related parameter is a chronological duration and/or an intensity of the event linked to the elevated substance emission via the oral-pharyngeal-nasal cavity of a vehicle occupant. If, for example, a vehicle occupant-related event is recognized (for example in reference to a qualitative presence of the event in the meaning of a yes/no determination), an event-related parameter can thus be determined in reference to this event. Multiple event-related parameters (determined by one or more identical or different sensors) can also be determined. The longer the duration of a possible vehicle occupant-related event (for example, coughing, sneezing)—associated with an elevated probability of transmitting pathogens—the greater the probability can be for transmitting pathogens to further vehicle occupants or the interior of the vehicle. A threshold can be established for a critical duration of certain vehicle occupant-related events above which an elevated probability of transmitting pathogens can be presumed. Critical limit values, from the exceeding of which an elevated probability of transmitting pathogens is to be expected, can also be established for the intensity (for example an amplitude value of a certain measured value or another characteristic feature of measurement data or measurement curves) of a certain vehicle occupant-related event—associated with an elevated probability of transmitting pathogens.

According to a further embodiment of the invention, it can be provided in a method according to the invention that the event-related parameter relates to a movement form, a movement duration, a movement intensity, or a movement sequence of a movement of the vehicle occupant. These parameters can also indicate an elevated probability of transmitting pathogens. Critical limit values, pattern movement forms, pattern movement sequences, etc. can also be predefined for these parameters, which can indicate an elevated probability of transmitting pathogens. For example, persons often carry out characteristic movement forms or movement sequences during coughing or sneezing. Such parameters can additionally also supply important items of information on the propagation direction, the probability of transmission to other occupants (distance between occupants, viewing direction of the occupants during the vehicle occupant-related event).

According to a further embodiment of the invention, it can be provided in a method according to the invention that the vehicle occupant-related parameter relates to a number, seat position, seat distribution, seat posture, height, age, weight, and/or a gender of the vehicle occupants. These parameters can also play an important role in the calculation of the probability of transmitting pathogens, in particular to establish whether a possible emission of pathogens by a vehicle occupant can result in an elevated probability of contamination of a further vehicle occupant. For example, seat positions of two vehicle occupants sitting opposite to one another frontally can be more critical when a vehicle occupant-related event (for example coughing or sneezing) prevails than when vehicle occupants face away from one another. The number of vehicle occupants also plays a role in the calculation of the probability of transmission. The fewer vehicle occupants are located in a vehicle interior, the lower the probability of infecting one or more vehicle occupants. The seat distribution (distances between the vehicle occupants) also plays an important role in the calculation of the probability of transmitting pathogens. The mentioned parameters are moreover relevant in the ascertainment of a possible measure for reducing the probability of transmitting pathogens.

According to a further embodiment of the invention, it can be provided in a method according to the invention that the vehicle-related parameter relates to an airflow parameter of an airflow situation present in the vehicle interior, wherein the airflow parameter is ascertained in particular from a vehicle speed, a window status, a door status, aeration settings, ventilation settings, climate control system settings, and/or air filter settings. The ascertainment of a vehicle-related parameter is relevant to ascertain an actual airflow situation in the vehicle interior. The actual airflow situation is a relevant influencing parameter for the probability of transmitting pathogens upon emission of pathogens to the ambient air (whether this is in the form of droplets or the like). If the actual airflow situation indicates a distribution of pathogens in the direction of further vehicle occupants, this can thus promote the probability of transmitting pathogens. In such a case, it is provided that the actual airflow situation is adapted in such a way that the probability of transmitting pathogens is reduced. The vehicle speed can have an effect on the aeration of the vehicle. A window status can mean that a window is closed, partially open, or completely open. The same applies to a door status, which can relate to a closed, partially open, or completely open door. Aeration settings can relate for example to the status (open, partially open, closed) of aeration slots or aeration openings. Ventilation settings can relate to a ventilation mode (e.g., suctioning in fresh air from the vehicle exterior, solely circulating interior air, etc.), the strength of a fan, the temperature of the air, etc. Climate control system settings can in particular relate to a set temperature. An air filter setting can in particular relate to a degree of contamination of an air filter or an air filter status in the meaning of “switched on” or “switched off”.

According to a further embodiment of the invention, it can be provided in a method according to the invention that the parameter tuple relates to a plurality of parameters which are selected from one or more event-related parameter(s), one or more vehicle occupant-related parameter(s), and one or more vehicle-related parameter(s). The parameters can be collected, stored, buffered, and evaluated on the vehicle-side or external computing and monitoring unit.

According to a further embodiment of the invention, it can be provided in a method according to the invention that the measure for reducing the probability of transmitting pathogens in the vehicle interior relates to adaptation of at least one vehicle parameter, wherein the adaptation relates in particular to an adaptation of an airflow situation present in the vehicle interior. It can be provided here that the adaptation of the at least one vehicle parameter relates to an adaptation of the vehicle speed, the window status, the door status, the aeration settings, the ventilation settings, the climate control system settings, and/or air filter settings. The adaptation preferably takes place based on an airflow situation present in a vehicle at a current point in time. The measure for reducing the probability of transmitting pathogens can also effect an information output, for example optically or acoustically, for example by flashing an optical warning signal, a speech output, or a display on a vehicle-side display screen, wherein the information output can also include that a driver is requested to stop or aerate the vehicle or to reduce the number of vehicle occupants.

The present invention (thus the method, system, and vehicle according to the invention) enables the recognition of a short-term abnormality (for example due to a vehicle occupant-related event such as coughing, sneezing event, or an elevated body temperature of a vehicle occupant established using a thermal imaging camera, for example), which can indicate elevated probability of transmitting pathogens. The recognition can in particular take place using one or more vehicle-side sensor(s) (as described above). All of the parameters, measurement data, etc. registerable according to the invention, in particular the vehicle occupant-related event, can be recorded in a time-resolved manner, for example by corresponding time recording devices (timers, clocks, etc.). The source of emitted pathogens and distribution in the vehicle interior can be ascertained using the sensors. The proportion or amount of emitted pathogens can also be determined according to the invention. Finally, the invention enables the initiation of adequate measures for reducing a probability of transmitting viruses on the basis of actual situation data registered at a current point in time.

### BRIEF DESCRIPTION OF THE DRAWINGS

Further features and advantages of the invention result from the following description of exemplary embodiments of the invention, which are to be understood as nonrestrictive and which are explained in more detail hereinafter with reference to the drawings. In the schematic figures of these drawings:

FIG. 1 shows a schematic method sequence of a method executable in the scope of the invention in a first embodiment variant; and

FIG. 2 shows a schematic method sequence of a method executable in the scope of the invention in a further embodiment variant.

### DETAILED DESCRIPTION

The invention firstly relates to a method for reducing a probability of transmitting pathogens between vehicle occupants located in a vehicle interior of a vehicle, in which a hazardous situation present in a vehicle interior of the vehicle is recognized, which is associated with an elevated probability of transmitting pathogens, namely i.) by registering a vehicle occupant-related event associated with the elevated probability of transmitting pathogens and/or ii.) by registering a current pathogen load in the vehicle interior and subsequently checking whether the registered current pathogen load is above a predefined threshold pathogen load, wherein the hazardous situation is present in the event of a current pathogen load lying above the threshold pathogen load, and/or iii.) on the basis of an instruction provided by a vehicle occupant or externally, which permits the elevated probability of transmitting pathogens and thus the hazardous situation to be concluded.

In the method, furthermore an event-related, vehicle occupant-related, and/or vehicle-related parameter or a parameter tuple is ascertained and, in the event of a recognized hazardous situation, a measure for reducing the probability of transmitting pathogens in the vehicle interior is initiated taking consideration of the ascertained parameter or parameter tuple.

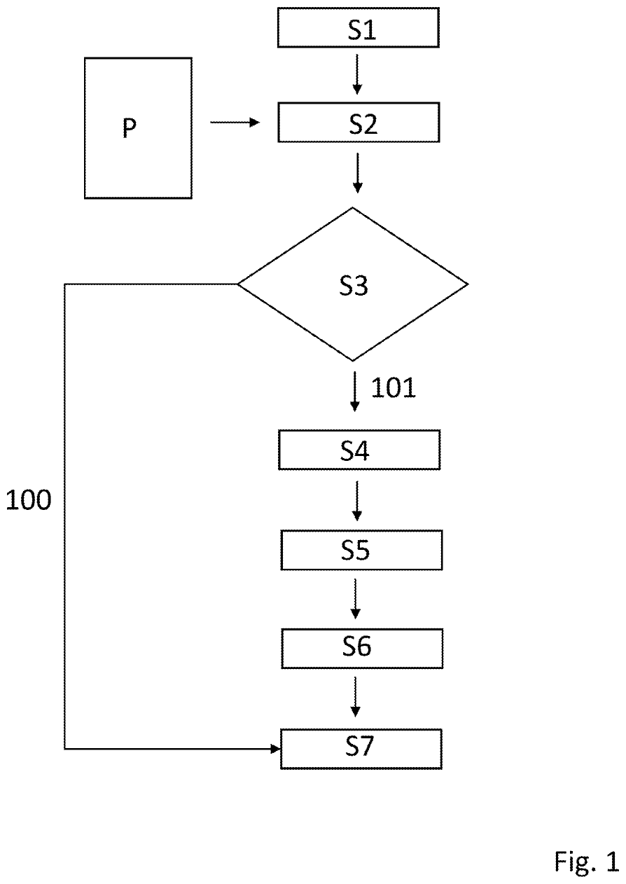

FIG. 1 shows a schematic method sequence of a method executed in the scope of the invention in a first embodiment variant. Here, after a start S1 of the method, in a step S2, one or more event-related, vehicle occupant-related, and/or vehicle-related parameters are ascertained by means of suitable sensors P. The ascertainment of the mentioned parameters can be triggered for example by a vehicle occupant-related event (for example a coughing or sneezing event) or can take place during the event. In a step S3, it is ascertained whether one of the parameters exceeds a certain parameter specification or a threshold value. For example, it can be checked whether the amount of the pathogens emitted by a vehicle occupant lies above a predefined threshold pathogen load. This can be carried out via a direct determination or an indirect determination (for example via parameters proportional to the pathogen load, which can be determined via different sensors P). If the parameter specification or the threshold value is not exceeded, for example in reference to the predefined threshold pathogen load, the method is thus ended according to flow line 100 (step S7). If the parameter specification or the threshold value is exceeded, for example in reference to the predefined threshold pathogen load, the method is thus continued according to flow line 101. In a step S4, for example a currently present pathogen load is then ascertained, for example via a direct determination or an indirect determination. Step S4 thus relates, in contrast to step S3 (qualitative determination in reference to a parameter specification or a threshold value), to a quantitative determination. In a step S5, the duration is calculated to reduce the probability of transmitting pathogens in the vehicle interior to an un-critical amount, or to ascertain the duration to purify the vehicle interior of the pathogen load. In step S6, a measure for reducing the probability of transmitting pathogens in the vehicle interior is then taken before the method ends in step S7. The measure for reducing the probability of transmitting pathogens preferably takes into consideration the ascertained event-related, vehicle occupant-related, and/or vehicle-related parameter(s) or a parameter tuple thereof.

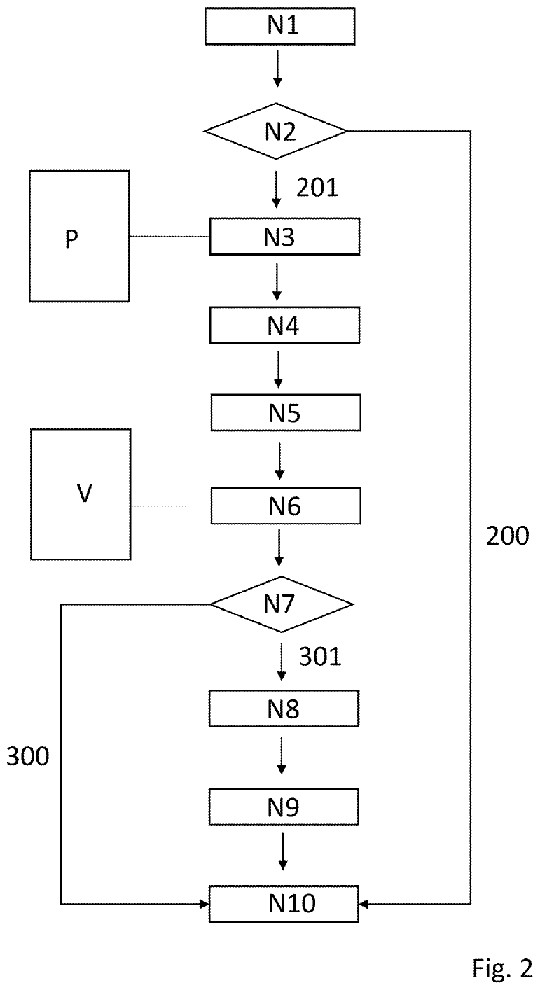

FIG. 2 shows a schematic method sequence of a method executed in the scope of the invention in a second embodiment variant.

If a (measurement) signal, an instruction, or other reference is present (step N1), which can indicate a possible hazardous situation associated with an elevated probability of transmitting pathogens, in step N2, the mentioned signal, the instruction, or the reference is checked in reference to the presence of an elevated probability of transmitting pathogens. If the signal, the instruction, or the reference does not supply an indication of an elevated probability of transmitting pathogens, the method is thus ended according to flow line 200 (step N10). Otherwise, according to flow line 201, sensor signals of the sensors P present in the vehicle are firstly checked in a step N3. In step N4, the source and direction of the pathogen propagation is checked based thereon. In step N5, it is checked how many vehicle occupants are located in the vehicle and at which spatial positions they are located in the vehicle. In step N6, airflow parameters relating to an airflow situation currently present in the vehicle interior are then ascertained, wherein the airflow parameter is in particular ascertained from parameters V such as a vehicle speed, a window status, a door status, aeration settings, ventilation settings, climate control system settings, and/or air filter settings. Thereupon, in step N7, the probability is ascertained according to which pathogens could be transmitted from one vehicle occupant to a further vehicle occupant. If the probability is low or a transmission is improbable, the method is thus ended according to flow line 300 (step N10). Otherwise, a measure is determined which can provide an airflow situation that is accompanied by a reduced probability of transmitting pathogens (step N8). In step N9, this measure is then initiated before the method ends in step N10.

When it is recognized according to the invention that a person coughs, sneezes, speaks with high intensity, or is infectious (vehicle occupant-related event), the position of the vehicle occupants, the location of the source, and the direction of the propagating infectious material are determined with the aid of the sensors and items of vehicle system information available in the vehicle. By changing the airflow settings, opening one or more windows, or activating air purification devices, the best airflow and air purification scenarios are ascertained and applied, so that pathogens are transported away from other vehicle occupants.

When a sensor signal is present that indicates a pathogen propagation in the vehicle interior, or such a propagation is recognized by manual actuation of a switch, sensors present in the vehicle are used and the measurement results thereof are combined to recognize the position of that vehicle occupant who coughs, sneezes, or speaks a large amount. In addition, it is registered whether the vehicle occupant has an elevated skin temperature, which can be an indicator of the presence of fever. The items of sensor information are used to ascertain the location of the source and the propagation direction as well as the position of other vehicle occupants who have to be protected. The items of information of the vehicle system (vehicle parameters) are used to assess the current airflow directions. If this would result in other vehicle occupants possibly inhaling or being contaminated by pathogens, an alternative airflow and purification setting is ascertained and set for a certain time period.

1. A method for reducing a probability of transmitting pathogens between vehicle occupants located in a vehicle interior of a vehicle, the method comprising:
determining a hazardous situation, which is associated with an elevated probability of transmitting pathogens, is present in the vehicle interior of the:
by registering a vehicle occupant-related event associated with the elevated probability of transmitting pathogens,
by registering a current pathogen load in the vehicle interior and subsequently checking whether the registered current pathogen load is above a predefined threshold pathogen load, wherein the hazardous situation is present in the event of a current pathogen load lying above the threshold pathogen load, and/or
on the basis of an instruction provided by a vehicle occupant or externally, which permits the elevated probability of transmitting pathogens and thus the hazardous situation to be concluded;
ascertaining an event-related, vehicle occupant-related, and/or vehicle-related parameter or a parameter tuple; and
in the event of a recognized hazardous situation, initiating a measure to reduce the probability of transmitting pathogens in the vehicle interior taking consideration of the ascertained parameter or parameter tuple.

2. The method according to claim 1, wherein the method is carried out using a vehicle-side computing and monitoring unit, in particular an onboard computer, or an external computing and monitoring unit connected in terms of communications technology to the vehicle-side computing and monitoring unit.

3. The method according to claim 1, wherein an event linked to an elevated substance emission via the oral-pharyngeal-nasal cavity of a vehicle occupant is registered as the vehicle occupant-related event.

4. The method according to claim 3, wherein the event is a coughing event, a sneezing event, a speech event, a voice event, a singing event, a respiratory event, a movement event, or a vomiting event, in which the substance is emitted.

5. The method according to claim 1, wherein an elevated body temperature of a vehicle occupant is registered as the vehicle occupant-related event.

6. The method according to claim 3, wherein the event is registered using one or more sensor(s), in particular one or more acoustic sensor(s), one or more optical sensor(s), one or more movement sensor(s), one or more thermal sensor(s), one or more radiation sensor(s), one or more seat sensor(s), one or more weight sensors arranged on a vehicle seat, and/or one or more touch sensor(s).

7. The method according to claim 1, wherein the current pathogen load is registered by a sensor arranged in the vehicle for determining a pathogen load in a room air sample or a room volume element from the vehicle interior.

8. The method according to claim 1, wherein that the instruction is based on a notification about a positive test of a pathogen infection of a vehicle occupant, wherein the instruction is transmitted in the course of a manual input of the vehicle occupant or automatically to the vehicle-side computing and monitoring unit.

9. The method according to claim 1, wherein the event-related parameter is a chronological duration and/or an intensity of the event linked to the elevated substance emission via the oral-pharyngeal-nasal cavity of a vehicle occupant.

10. The method according to claim 1, wherein the event-related parameter relates to a movement form, a movement duration, a movement intensity, or a movement sequence of a movement of a vehicle occupant.

11. The method according to claim 1, wherein the vehicle occupant-related parameter relates to a number, seat position, seat distribution, seat posture, height, age, weight, and/or a gender of the vehicle occupants.

12. The method according to claim 1, wherein the vehicle-related parameter relates to an airflow parameter of an airflow situation present in the vehicle interior, wherein the airflow parameter is ascertained in particular from a vehicle speed, a window status, a door status, aeration settings, ventilation settings, climate control system settings, and/or air filter settings.

13. The method according to claim 1, wherein the parameter tuple relates to a plurality of parameters which are selected from one or more event-related parameter(s), one or more vehicle occupant-related parameter(s), and/or one or more vehicle-related parameter(s).

14. The method according to claim 1, wherein the measure for reducing the probability of transmitting pathogens in the vehicle interior relates to an adaptation of at least one vehicle parameter, wherein the adaptation relates in particular to an adaptation of an airflow situation present in the vehicle interior.

15. The method according to claim 14, wherein the adaptation of the at least one vehicle parameter relates to an adaptation of the vehicle speed, the window status, the door status, the aeration settings, the ventilation settings, the climate control system settings, and/or air filter settings.

16. A system implemented in a vehicle, the system comprising:
a computing device configured to:
determine a hazardous situation present in a vehicle interior of the vehicle, which is associated with an elevated probability of transmitting pathogens, comprising:
registration means for registering a vehicle occupant-related event associated with the elevated probability of transmitting pathogens,
pathogen load registration means for registering a current pathogen load in the vehicle interior and checking means for checking whether the registered current pathogen load is above a predefined threshold pathogen load, wherein the hazardous situation is present in the event of a current pathogen load lying above the threshold pathogen load, and/or
receiving means for receiving an instruction provided by a vehicle occupant or externally, from which the elevated probability of transmitting pathogens and thus the hazardous situation can be concluded;
ascertain an event-related, vehicle occupant-related, and/or vehicle-related parameter or a parameter tuple; and
initiate a measure for reducing the probability of transmitting pathogens in the vehicle interior taking consideration of the ascertained parameter or parameter tuple in the event of an established hazardous situation.

17. A vehicle comprising a system having the features of claim 16.
